(19) United States
(12) Patent Application Publication
(10) Pub. No.: US 2024/0335766 A1
(43) Pub. Date: Oct. 10, 2024

(54) APPARATUS AND METHOD FOR REMOVING BUBBLES FROM FLUID SAMPLE WITH SLIDABLE FILTER

(71) Applicant: Siemens Healthcare Diagnostics Inc., Tarrytown, NY (US)

(72) Inventors: Janine Cox, Stoughton, MA (US); Justin DeSilva, Mendon, MA (US)

(73) Assignee: Siemens Healthcare Diagnostics Inc., Tarrytown, NY (US)

(21) Appl. No.: 18/681,788

(22) PCT Filed: Aug. 5, 2022
(86) PCT No.: PCT/US2022/074577

§ 371 Date: Feb. 6, 2024

Related U.S. Application Data
(60) Provisional application No. 63/232,365, filed on Aug. 12, 2021.

(51) Int. Cl. B01D 19/00; B01D 19/02; B01D 71/26; B01D 71/36

(52) U.S. Cl. CPC B01D 19/0031; B01D 19/02; B01D 71/261; B01D 71/262; B01D 71/36; B01D 2325/20

(57) ABSTRACT

An apparatus and method for removing bubbles from a fluid sample. The apparatus has a barrel and a filter member. The barrel has a first end, a second end, a sidewall, and an inner surface defining an internal chamber. The first side has an inlet opening and the second side has an outlet opening. The filter member is disposed within the internal chamber and defines an inlet side and an outlet side of the internal chamber. The filter member has a gas-permeable, liquid-impermeable membrane which permits the gas portion of the fluid sample to pass across the filter member from the inlet side to the outlet side. The gas-permeable, liquid-impermeable membrane provides a fluid-tight seal across the filter member. The filter member is pierceable so a probe may be passed through the filter member to withdraw the liquid portion of the fluid sample from the internal chamber.

### CROSS-REFERENCE TO RELATED APPLICATIONS

This application claims benefit under 35 USC § 119 (e) of U.S. Provisional Application No. 63/232,365, filed Aug. 12, 2021. The entire contents of the above-referenced patent application(s) are hereby expressly incorporated herein by reference.

### BACKGROUND

Blood sampling is a common health care procedure typically used in hospital and laboratory settings to determine the physiological and biochemical condition of a patient. Blood sampling is essential to the diagnosis and treatment of patients suspected of a wide variety of disorders. Blood samples are analyzed by fluid testing devices, such as blood analyzers, to detect clinically significant variations in blood components, e.g., plasma, red blood cells, white blood cells, and platelets, or other characteristics, such as blood gas conditions. Analysis of a blood sample for blood gas conditions provides information regarding the amount of oxygen and carbon dioxide in the blood, and may also be used to determine the pH of a blood sample. Imbalances in the oxygen, carbon dioxide, or PH levels of a blood sample may indicate particular pathological conditions or stage of disease progression.

Blood samples are typically collected using blood collection syringes having hypodermic needles, or vacuum tubes coupled to a needle assembly. However, blood collection syringes are prone to have bubbles of air or other gases within the syringe. The bubbles in the barrel and tip of the syringe may interfere with the analysis of blood samples. Regarding blood gas analysis, bubbles trapped in a syringe may cause a blood analyzer to produce erroneous results. To obtain accurate results, blood samples need to be mixed thoroughly and all air must be expelled before mixing. Bubbles are typically expelled by tapping the sides of the syringe to force the air or gas to the top of the syringe. Once the bubbles have reached the top of the syringe, a piece of gauze or tissue is put over the top to force the gas (and some blood) out of the syringe. This method poses not only a biohazard risk because the blood sample is freely flowing out of the top of the syringe, but also an exposure risk, as the tip of the syringe is still open to ambient air. A blood analyzer analyzing a blood sample collected in this manner risks producing erroneous results.

Further, blood analyzers often may not permit direct sample input via a syringe or vacuum tube. In that case, the syringe and/or vacuum tube is merely an intermediate container for the blood sample, and at least a portion of the blood sample must be removed and transferred to a secondary sample container capable of being accommodated by the blood analyzer. Transferring the blood sample to a secondary sample container presents its own exposure risks and may increase the likelihood of obtaining erroneous results.

A need exists for an apparatus and method for removing bubbles from a fluid sample. It is to such an apparatus and method that the inventive concepts disclosed and claim herein are directed.

### SUMMARY OF THE INVENTIVE CONCEPTS

The inventive concepts disclosed and claimed herein generally relate to an apparatus for removing bubbles from a fluid sample having a liquid portion and a gas portion. The apparatus includes a barrel and a filter member. The barrel has a first end, a second end, a sidewall, and an inner surface. The sidewall extends between the first end and the second end of the barrel, and the inner surface of the barrel defines an internal chamber. The first end of the barrel has an inlet opening and the second end has an outlet opening. The filter member is disposed within the internal chamber so the filter member defines an inlet side and an outlet side of the internal chamber. The filter member is positioned between the first end and the second end of the filter member. The filter member has at least one gas-permeable, liquid-impermeable membrane to permit at least a portion of the gas portion of the fluid sample to pass across the filter member from the inlet side to the outlet side of the internal chamber. The at least one gas-permeable, liquid-impermeable membrane provides a fluid-tight seal cross the filter member to prevent the liquid portion of the fluid sample from passing from the inlet side to the outlet side as the fluid sample is passed into the internal chamber via the inlet opening to separate at least a portion of the gas portion from the liquid portion of the fluid sample. The filter member is pierceable so a probe may be passed through the filter member from the outlet side to the inlet side to withdraw the liquid portion of the fluid sample from the inlet side of the internal chamber.

### BRIEF DESCRIPTION OF THE DRAWINGS

To assist those of ordinary skill in the relevant art in making and using the inventive concepts disclosed herein, reference is made to the appended drawings and schematics, which are not intended to be drawn to scale, and in which like reference numerals are intended to refer to the same or similar elements for consistency. For purposes of clarity, not every component may be labeled in every drawing. Certain features and certain views of the figures may be shown exaggerated and not to scale or in schematic in the interest of clarity and conciseness. In the drawings:

FIG. 1 is a side perspective view of an exemplary embodiment of an apparatus for removing bubbles according to the inventive concepts disclosed herein shown coupled to a sample receiving assembly.

FIG. 2A is a longitudinal, cross-sectional view of the apparatus of FIG. 1 coupled to a collection syringe illustrating positioning of a filter member and a plunger assembly prior to removal of bubbles from the fluid sample.

FIG. 2B is a longitudinal, cross-sectional view of the apparatus of FIG. 1 coupled to a collection syringe illustrating positioning of the filter member and plunger assembly following removal of bubbles from the fluid sample.

FIG. 2C is a longitudinal, cross-sectional view of the apparatus of FIG. 1 coupled to a collection syringe illustrating the insertion of a probe into the apparatus following removal of bubbles from of the fluid sample.

FIG. 3A is a longitudinal, cross-sectional view of the apparatus of FIG. 1 illustrating a position of the filter member prior to removal of bubbles from the fluid sample.

FIG. 3B is a is a longitudinal, cross-sectional view of the apparatus of FIG. 1 illustrating a position of the filter member following removal of bubbles from the fluid sample.

FIG. 3C is a longitudinal, cross-sectional view of the apparatus of FIG. 1 illustrating the insertion of the probe into the apparatus following removal of bubbles from the fluid sample.

FIG. 4 is a perspective view of the apparatus.

FIG. 5 is an exploded, cross-sectional view of the apparatus.

FIG. 6A is a perspective view of an exemplary embodiment of the filter member according to the inventive concepts disclosed herein.

FIG. 6B is a cross-sectional view taken along line 6B-6B of FIG. 6A.

FIG. 7 is a perspective view of another embodiment of a filter member according to the inventive concepts disclosed herein.

FIG. 8A is a perspective view of another embodiment of a filter member according to the inventive concepts disclosed herein.

FIG. 8B is a cross-sectional view taken along line 8B-8B of FIG. 8A

### DETAILED DESCRIPTION OF EXEMPLARY EMBODIMENTS

Before explaining at least one embodiment of the inventive concept(s) in detail by way of exemplary drawings, experimentation, results, and laboratory procedures, it is to be understood that the inventive concept(s) is not limited in its application to the details of construction and the arrangement of the components set forth in the following description or illustrated in the drawings, experimentation and/or results. The inventive concept(s) is capable of other embodiments or of being practiced or carried out in various ways. The language used herein is intended to be given the broadest possible scope and meaning; and the embodiments are meant to be exemplary-not exhaustive. Also, it is to be understood that the phraseology and terminology employed herein is for the purpose of description and should not be regarded as limiting.

Unless otherwise defined, scientific and technical terms used in connection with the presently disclosed and claimed inventive concept(s) shall have the meanings commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All the articles, compositions and/or methods disclosed and claimed herein can be made and executed without undue experimentation, given the present disclosure. While the articles, compositions and methods of the inventive concept(s) have been described in terms of preferred embodiments, it will be apparent to those of skill in the art that variations may be applied to the articles, compositions and/or methods and in the steps or in the sequence of steps of the methods described herein without departing from the concept, spirit and scope of the inventive concept(s). All such similar substitutes and modifications apparent to those skilled in the art are deemed to be within the spirit, scope and concept of the inventive concept(s) as defined by the appended claims.

As utilized under the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the inherent variation of error for the device, the method being employed to determine the value, or the variation that exists among the study subjects.

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, the term “sample” and variations thereof is intended to include biological tissues, biological fluids, chemical fluids, chemical substances, suspensions, solutions, slurries, mixtures, agglomerations, tinctures, slides, powders, or other preparations of biological tissues or fluids, synthetic analogs to biological tissues or fluids, bacterial cells (prokaryotic or eukaryotic), viruses, single-celled organisms, lysed biological cells, fixed biological cells, fixed biological tissues, cell cultures, tissue cultures, genetically engineered cells and tissues, genetically engineered organisms, and combinations thereof, for example.

In the following detailed description of embodiments of the inventive concept, numerous specific details are set forth in order to provide a more thorough understanding of the inventive concept. However, it will be apparent to one of ordinary skill in the art that the inventive concept within the disclosure may be practiced without these specific details. In other instances, well-known features have not been described in detail to avoid unnecessarily complicating the instant disclosure.

Finally, as used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Described herein, and shown in the accompanying figures, are several non-limiting embodiments of apparatus of the presently claimed and disclosed inventive concepts which may be used in association with collection syringes and fluid sample analyzers for removing bubbles of air or other gases from a fluid sample for analysis by a fluid sample analyzer. The fluid sample is generally from a biological source. A “fluid” refers to any substance that has no fixed shape and yields easily to external pressure.

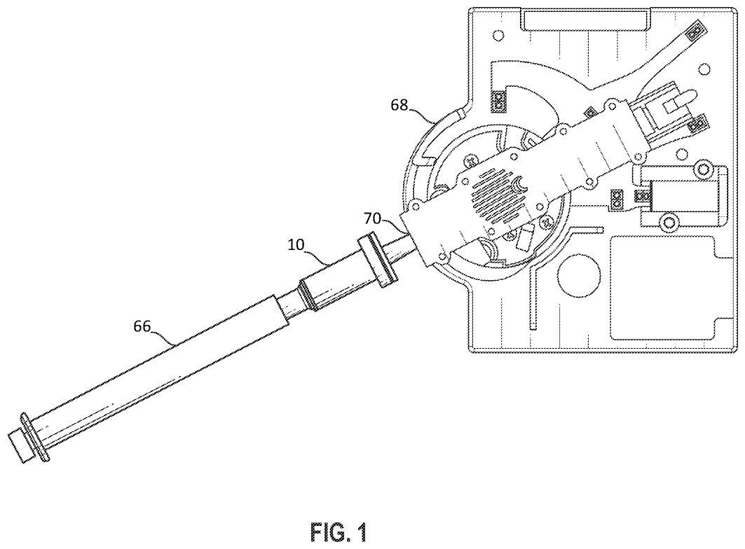

Referring now to the drawings, and more particularly to FIGS. 1-5, shown therein is an exemplary embodiment of an apparatus 10 for removing bubbles from a fluid sample constructed in accordance with the inventive concepts disclosed and claimed herein. The apparatus 10 includes a barrel 12, a nozzle cap 14, and a filter member 16.

The barrel 12 includes a first end 18, a second end 20, a sidewall 22, and an inner surface 24. The barrel 12 may be of any suitable size and shape, and formed of any suitable material, such as, without limitation, plastics such as polycarbonate, polystyrene, polyacrylates, and polyurethane, or medical-grade polymers. The sidewall 22 of the barrel 12 extends between the first end 18 and the second end 20 of the barrel 12. The inner surface 24 of the barrel 12 defines an internal chamber 26. The first end 18 has an inlet opening 28 and the second end 20 has an outlet opening 30.

The internal chamber 26 may be of any suitable size and shape to contain a fluid sample 32. The fluid sample 32 may be, for example, blood, serum, plasma, or other bodily fluids. The fluid sample 32 may contain a gas portion and a liquid portion. The gas portion of the fluid sample 32 may be, for example, air or other gases. A portion of the gas portion may form bubbles in the fluid sample

The inlet opening 28 and the outlet opening 30 may have a cross-section of any suitable geometry, including, but not limited to, circular, oval, square, or rectangular. The inlet opening 28 and the outlet opening 30 may be molded or cut into the barrel 12, or otherwise pre-fabricated. The inlet opening 28 may be formed to prevent coagulation as the fluid sample 32 is passed into the internal chamber 26 via the inlet opening 28.

The outlet opening 30 may be provided with a nozzle cap 14. The nozzle cap 14 includes an annular wall 36 and a tubular portion 38 having a bore 40 extending therethrough. The bore 40 may have a cross-section of any suitable geometry, including, but not limited to, circular, oval, square, or rectangular. The bore 40 may be sized to have a diameter adapted to slidably axially receive a sample probe. The base of the tubular portion 38 flares outwardly and merges with the annular wall 36 at a rim 42, the annular wall 36 tapers downwardly to form an inverted frusto-conical section. The nozzle cap 14 may be releasably coupled to the outlet opening 30 such that the bore 40 is aligned with the outlet opening 30 to permit fluid communication with the internal chamber 26.

The filter member 16 is disposed within the internal chamber 26 so the filter member 16 defines an inlet side 44 and an outlet side 46 of the internal chamber 26. The filter member 16 is positionable between the first end 18 and the second end 20 of the barrel 12. The filter member 16 includes at least one gas-permeable, liquid-impermeable membrane 48. The filter member 16 may be any suitable shape and size to sealingly engage the inner surface 24 of the barrel 12. The filter member 16 may be formed of any suitable material, such as, without limitation, a rubber, an elastomer, a polyolefin-based resin, a fluorine-based resin, or a polyester-based resin. The elastomer may include, for example, a polyvinyl chloride-based elastomer, a polyolefin-based elastomer, a styrene-based elastomer, a polyester-based elastomer, a polyamide-based elastomer, a polyurethane-based elastomer, or a mixture thereof.

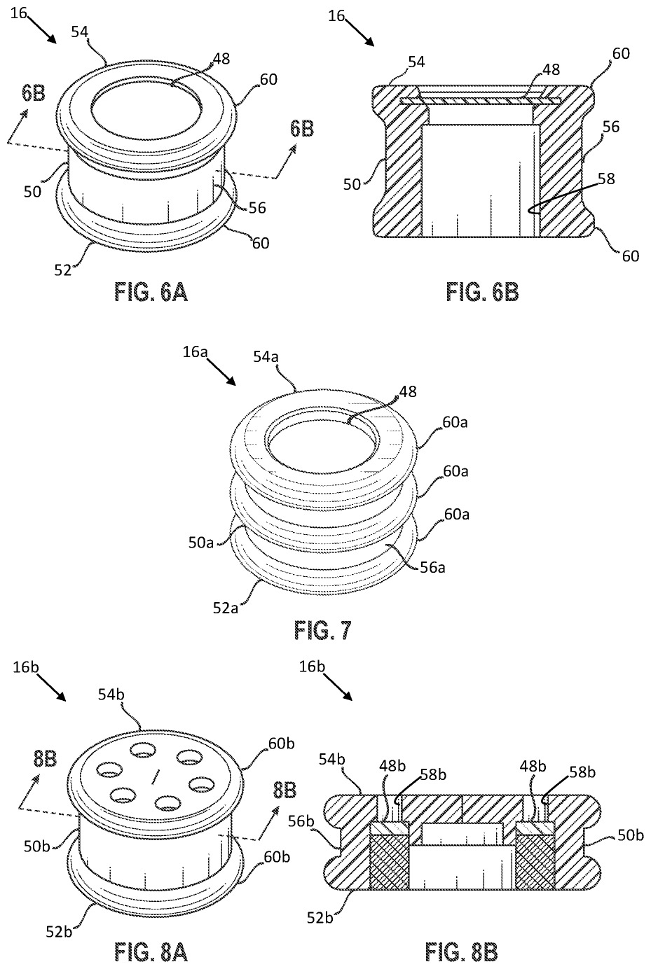

Referring now to FIGS. 6A and 6B, shown therein is a perspective and cross-sectional view, respectively, of an exemplary embodiment of the filter member 16. The filter member 16 comprises a body 50 having a first end 52, a second end 54, and a sidewall 56 extending from the first end 52 to the second end 54. The sidewall 56 of the body 50 defines a passageway 58 extending through the body 50 from the first end 52 to the second end 54. The sidewall 56 of the body 50 may have at least two annular projections 60 extending radially outwardly in slidable, sealing contact with the inner surface 24 of the barrel 12. As shown in FIGS. 6A and 6B, the body 50 may have two annular projections 60 spaced apart from each other. The annular projections 60 may include convex or concave features.

The filter member 16 further includes a gas-permeable, liquid-impermeable membrane 48 which extends across the entirety of the passageway 58. In one embodiment, the gas-permeable, liquid-impermeable membrane 48 is secured to the body 50 adjacent the second end 54 of the body 50, as shown in FIG. 6B.

Referring now to FIG. 7, shown therein is a perspective view of another embodiment of a filter member 16a constructed in accordance with the inventive concepts disclosed and claimed herein. Similar to previously described embodiments, the filter member 16a comprises a body 50a having a first end 52a, a second end 54a, and a sidewall 56a extending from the first end 52a to the second end 54a. The sidewall 56a of the body 50a defines a passageway 58a (not shown) extending through the body 50a from the first end 52a to the second end 54a. As in FIGS. 6A and 6B of the previously described embodiment, the body 50a may have at least two annular projections 60b extending radially outwardly in slidable, sealing contact with the inner surface 24 of the barrel 12. As shown in FIG. 7, the body 50a may have three annular projections 60a spaced apart from each other. The filter member 16a further includes the gas-permeable, liquid-impermeable membrane 48 which extends across the entirety of the passageway 58a. In this embodiment, the gas-permeable, liquid-impermeable membrane 48 is secured to the body 50a adjacent the second end 54a of the body.

Referring now to FIGS. 8A and 8B, shown therein is a perspective and cross-sectional view, respectively, of another embodiment of a filter member 16b. The filter member 16b comprises a body 50b having a first end 52b, a second end 54b, and a sidewall 56b extending from the first end 52b to the second end 54b. Similar to previously described embodiments, the body 50b may have at least two annular projections 50b extending radially outwardly in slidable, sealing contact with the inner surface 24 of the barrel 12. The sidewall 56b of the body 50b defines a plurality of passageways 58b extending through the body 50b from the first end 52b to the second end 54b. In that embodiment, the plurality of passageways 58b may be in a parallel relationship to one another, as shown in FIG. 8B. Further, the filter member 16b may include a plurality of gas-permeable, liquid-impermeable membranes 48b with at least one of the gas-permeable, liquid-impermeable membranes 48b extending across each of the plurality of passageways 58b of the body 50b. The filter member 16b may further include a plurality of porous filter material 62 positioned between the first end 52b of the body 50b and each of the plurality of gas-permeable, liquid-impermeable membranes 48b to prevent solid particulate from contacting the plurality of gas-permeable, liquid-impermeable membranes 48b, as shown in FIG. 8B.

The at least one gas-permeable, liquid-impermeable membrane 48 may be formed of any suitable material, such as, without limitation, polytetrafluoroethylene, polyvinylidene fluoride, polyvinylchloride, polyolefins like polypropylene, polyethylene, polymethylpentene, polyamides, polysulfones, polyetheretherketones, polycarbonates, and combinations including any of these. In one embodiment, the gas-permeable, liquid-impermeable membrane 48 is formed from a material comprising at least one of polytetrafluorethylene, polypropylene, and polyethylene. The at least one gas-permeable, liquid-impermeable membrane 48 may have a thickness suitable for allowing puncture upon application of a mechanical force. The at least one gas-permeable, liquid-impermeable membrane 48 may permit at least a portion of the gas portion of the fluid sample 32, which forms bubbles, to pass across the filter member 16 from the inlet side 44 to the outlet side 46 of the internal chamber 26. The at least one gas-permeable, liquid-impermeable membrane 48 also provides a fluid-tight seal across the filter member 16 to prevent the liquid portion of the fluid sample 32 from passing from inlet side 44 to the outlet side 46 as the fluid sample 32 is passed into the internal chamber 26 via the inlet opening 28 to separate at least a portion of the gas portion from the liquid portion of the fluid sample 32. The filer member 16 is pierceable so a sample probe 72 may be passed through the filter member 16 from the outlet side 46 to the inlet side 44 to withdraw the liquid portion of the fluid sample 32 from the inlet side 44 of the internal chamber 26.

As shown in FIG. 1, the apparatus 10 may be used in association with a collection syringe 66 and a fluid sample analyzer 68 to remove bubbles from the fluid sample 32 having a liquid portion and a gas portion. Although, FIG. 1 shows the apparatus 10 associated with the collection syringe 66 and the fluid sample analyzer 68, those skilled in the art will understand and appreciate that the apparatus 10 may independently be associated with collection devices other than the collection syringe 66, such as, for example, a vacuum tube, and medical devices other than the fluid sample analyzer 68. The fluid sample analyzer 68 may be any suitable fluid testing device, such as, without limitation, microfluidic devices, blood gas analyzers, hematology analyzers, urine chemistry analyzers, and the like.

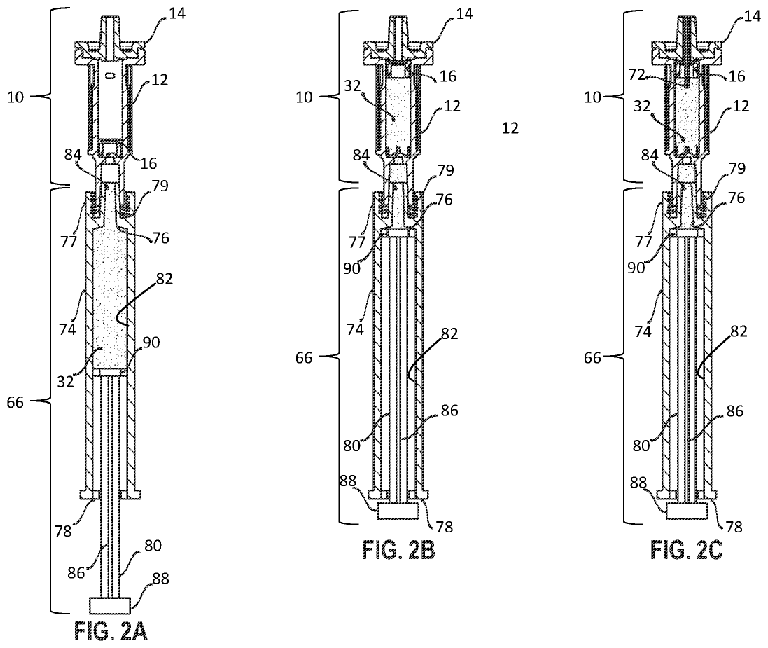

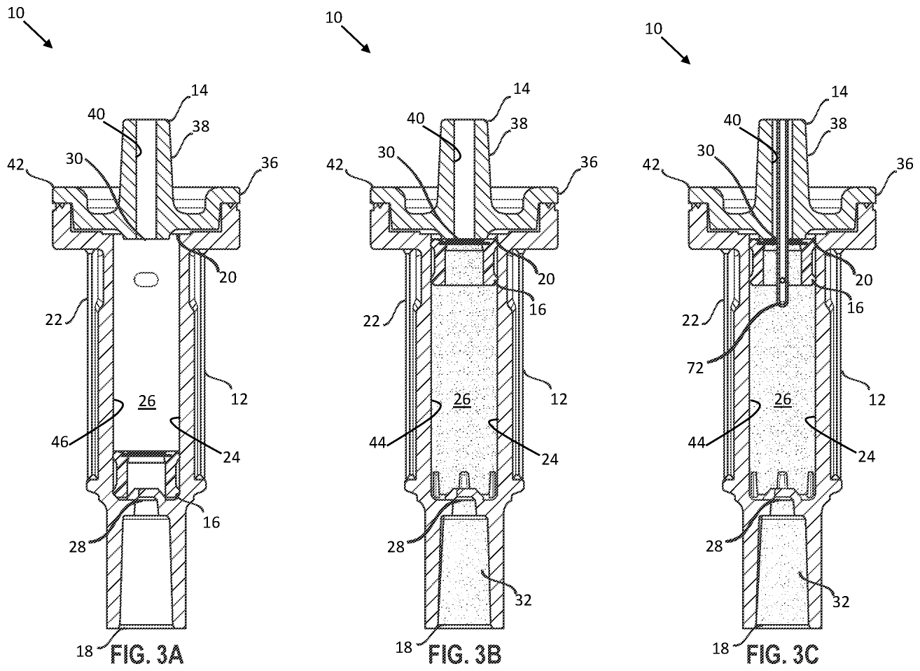

The fluid sample analyzer 68 includes a sample input port 70 and a sample probe 72. The sample probe 72 may be axially slidable relative to the sample input port 70. The sample input port 70 may be sized to receive and detachably secure at least a portion of the nozzle cap 14, as shown in FIG. 1. The sample probe 72 may be extended to pass through the filter member 16 from the outlet side 46 of the internal chamber 26 to the inlet side 44 to withdraw at least a portion of the fluid sample 32 into the fluid sample analyzer 68 from the inlet side 44 of the internal chamber 26, as shown in FIGS. 2C and 3C. The sample probe 72 may be of a length compatible with sampling from the inlet side 44 of the internal chamber 26.

The collection syringe 66 includes a syringe body 74 having a front end 76, a rear end 78, and a plunger 80. The syringe body 74 defines a reservoir 82 within which the fluid sample 32 may be contained and later expelled via a dispensing opening 84 positioned at the front end 76 of the syringe body 74. The rear end 78 of the syringe body 74 may be open and provided with a body flange 85 to facilitate the collection and expulsion of the fluid sample 32. The syringe body 74 may be any suitable size or shape for collection of fluid samples, such as, for example, a cylindrical shape. The syringe body 74 may also include a collar 77 formed concentrically with the dispensing opening 84 into a cylindrical shape to surround the dispending opening 84. The collar 77 may include an inner peripheral surface in which a threaded engagement portion 79 is formed for engaging the apparatus 10. The syringe body 74 may be constructed of any suitable material, such as glass or plastic. The syringe body 74 may have an outer diameter adapted to coaxially slide within inlet opening 28 of the apparatus 10.

The plunger 80 may include a shaft 86 that terminates at one end in a plunger flange 88 to facilitate the collection and expulsion of the fluid sample 32. The shaft 86 may, for example, have a cylindrical shape or a columnar shape, and may have a cross-section of a polygonal shape, such as a square, pentagonal, hexagonal, or cruciform shape. The plunger 80 may further include a plunger seal 90 secured to the shaft 86 opposite the plunger flange 88. The plunger 80 may be removably disposed within the syringe body 74 and may be selectively movable within the reservoir 82. The plunger seal 90 has a diameter that permits the plunger seal 90 to create a fluid-tight seal when positioned within the reservoir 82 such that the liquid sample 32 may not move past the plunger seal 90. Further, the plunger seal 90 prevents ambient air from moving from the rear end 78 of the syringe body 74 in a direction past the plunger seal 90. The plunger 80 may be axially displaced relative to the syringe body 74. Movement of the plunger 80 from the rear end 78 to the front end 76 of the syringe body 74 may cause at least a portion of the fluid sample 32 to be expelled from the reservoir 82 and introduced into the inlet opening 28 of the apparatus 10 via the dispensing opening 84. The plunger 80 may be constructed of any suitable polymeric material known in the art.

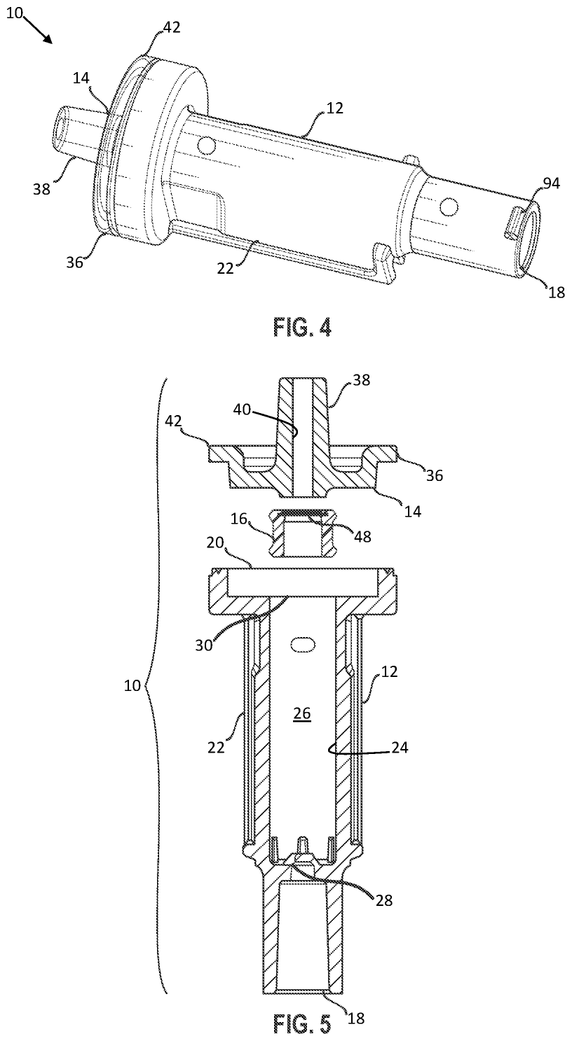

To remove the gas portion (i.e., bubbles) of the fluid sample 32 from the liquid portion, the collection syringe 66, containing a volume of the fluid sample 32 within the reservoir 82, is releasably attached to the inlet opening 28 of the barrel 12, as shown in FIG. 2A. As shown in FIGS. 2A-2C, the front end 76 of the syringe body 74 may be releasably connected to the apparatus 10 by way of the threaded engagement portion 79. As shown in FIG. 4, the barrel 12 may include a barrel connection portion 94. In one embodiment, the threaded engagement portion 79 of the syringe body 74 is a male luer connection and the barrel connection portion 94 is a female luer connector, although other suitable connectors may be utilized. As shown in FIGS. 2A-2C, the threaded engagement portion 79 may detachably engage the barrel connection portion 94 such that significant relative movement between the collection syringe 66 and the apparatus 10 is prevented, and fluid communication between the reservoir 82 and the inlet opening 28 of the apparatus 10 is permitted.

The collection syringe 66 and the apparatus 10 are positioned in an upright orientation with the apparatus 10 above the collection syringe 66 and the bubbles in the fluid sample rise to the top of the fluid sample. The plunger 80 of the collection syringe 66 is displaced axially along the reservoir 82 a distance from the rear end 78 towards the front end 76 of the syringe body 74, as shown in FIG. 2A. Movement of the plunger 80 within the reservoir 82 causes at least a portion of the gas portion (i.e., bubbles) of the fluid sample 32 to be expelled from the reservoir 82 and into the internal chamber 26 of the apparatus 10 via the inlet opening 28, passing through the filter member 16. The gas portion of the fluid sample 32 passes through the filter member 16, and is then ultimately expelled from the internal chamber 26 of the apparatus 10. Once, at least a portion of the gas portion of the fluid sample 32 has been displaced from the reservoir 82, the plunger 80 experiences an initial resistive force.

Upon application of sufficient force to overcome the initial resistive force, the plunger 80 is advanced further into the reservoir 82 towards the front end 76 of the syringe body 74, as shown in FIG. 2B, thereby increasing the internal pressure of the reservoir 82. As the internal pressure of the reservoir 82 increases, it produces a force sufficient to cause at least a portion of the liquid portion of the fluid sample 32 to be expelled from the reservoir 82 and into the internal chamber 26 of the barrel 12 via the inlet opening 28. The fluid sample 32 entering the internal chamber 26 may cause the filter member 16 to be displaced axially along the internal chamber 26 towards the second end 20 of the barrel 12, as shown in FIGS. 2B and 3B. The filter member 16 may be displaced such that it becomes disposed adjacent the outlet opening 30. This arrangement prevents ambient air from entering the internal chamber 26 and prevents the fluid sample 32 from exiting the internal chamber 26 via the outlet opening 30. In some embodiments, the plunger 80 may be partially extended into the reservoir 82 so less than all of the fluid sample 32 is transferred from the reservoir 82 into the internal chamber 26.

Once the liquid portion of the fluid sample 32 has been expelled from the reservoir 82 and is contained within the internal chamber 26 of the apparatus 10, the sample probe 72 of the fluid sample analyzer 68 may be extended from the sample input port 70 and passed through the filter member 16 to withdraw the liquid portion of the fluid sample 32 from the inlet side 44 of the internal chamber 26, as shown in FIGS. 2C and 3C. In one embodiment, the sample probe 72 pierces the gas-permeable, liquid-impermeable membrane 48 of the filter member 16 to gain fluid access to the inlet side 44 of the internal chamber 26.

From the above description, it is clear that the inventive concept(s) disclosed herein is well adapted to carry out the objects and to attain the advantages mentioned herein as well as those inherent in the inventive concept disclosed herein. While exemplary embodiments of the inventive concept disclosed herein have been described for purposes of this disclosure, it will be understood that numerous changes may be made which will readily suggest themselves to those skilled in the art and which are accomplished without departing from the scope of the inventive concept disclosed herein and defined by the appended claims.

The following is a list of non-limiting illustrative embodiments of the inventive concepts disclosed herein:

An illustrative apparatus for removing bubbles from a fluid sample having a liquid portion and a gas portion, comprising:
a barrel having a first end, a second end, a sidewall extending between the first end and the second end, and an inner surface defining an internal chamber, the first end having an inlet opening and the second end having an outlet opening; and
a filter member disposed within the internal chamber so the filter member defines an inlet side and an outlet side of the internal chamber and so the filter member is positionable between the first end and the second end of the barrel, the filter member having a gas-permeable, liquid-impermeable membrane to permit at least a portion of the gas portion of the fluid sample to pass across the filter member from the inlet side to the outlet side of the internal chamber and to provide a fluid-tight seal across the filter member to prevent the liquid portion of the fluid sample from passing from the inlet side to the outlet side as the fluid sample is passed into the internal chamber via the inlet opening to separate at least a portion of the gas portion from the liquid portion of the fluid sample;
wherein the filter member is pierceable so a probe may be passed through the filter member from the outlet side to the inlet side to withdraw the liquid portion of the fluid sample from the inlet side of the internal chamber.

The illustrative apparatus wherein the filter member further comprises a body having a first end, a second end, and a sidewall extending from the first end to the second end, the sidewall of the body defining a passageway extending through the body from the first end to the second end, and wherein the gas-permeable, liquid-impermeable membrane extends across an entirety of the passageway.

The illustrative apparatus wherein the gas-permeable, liquid-impermeable membrane is secured to the body adjacent the second end of the body.

The illustrative apparatus wherein the gas-permeable, liquid-impermeable membrane extends across an entirety of the passageway at the second side.

The illustrative apparatus wherein the filter member is slidably disposed in the internal chamber of the barrel.

The illustrative apparatus wherein the sidewall of the body has at least two annular projections extending radially outwardly in slidable, sealing contact with the inner surface of the barrel.

The illustrative apparatus wherein the gas-permeable, liquid-impermeable membrane is formed from a material comprising at least one of polytetraflyorethylene, polypropylene, and polyethylene.

The illustrative apparatus wherein the body has a plurality of passageways extending through the body from the first end to the second end, and wherein the filter member further has a plurality of gas-permeable, liquid impermeable membranes with at least one of the gas-permeable, liquid impermeable membranes extending across each of the passageways of the body.

The illustrative apparatus wherein the filter member further comprises a plurality of porous filter material positioned between the first end of the body and each of the gas-permeable, liquid impermeable membranes to prevent solid particulate from contacting the gas-permeable, liquid impermeable membranes.

The illustrative apparatus wherein the plurality of gas-permeable, liquid-impermeable membranes are secured to the body adjacent the second end of the body.

The illustrative apparatus wherein the passageways are in a parallel relationship to one another.

An illustrative apparatus for removing bubbles from a fluid sample having a liquid portion and a gas portion in combination with a liquid sample analyzer having a sample probe, comprising:
a barrel having a first end, a second end, a sidewall extending between the first end and the second end, and an inner surface defining an internal chamber, the first end having an inlet opening and the second end having an outlet opening; and
a filter member disposed within the internal chamber so the filter member defines an inlet side and an outlet side of the internal chamber and so the filter member is positionable between the first end and the second end of the barrel, the filter member having a gas-permeable, liquid-impermeable membrane to permit at least a portion of the gas portion of the fluid sample to pass across the filter member from the inlet side to the outlet side of the internal chamber and to provide a fluid-tight seal across the filter member to prevent the liquid portion of the fluid sample from passing from the inlet side to the outlet side as the fluid sample is passed into the internal chamber via the inlet opening to separate at least a portion of the gas portion from the liquid portion of the fluid sample;
wherein the filter member is pierceable by the sample probe so the sample probe may be passed through the outlet opening of the barrel and through the filter member from the outlet side to the inlet side to draw the liquid portion of the fluid sample into the liquid sample analyzer from the inlet side of the internal chamber.

The illustrative combination wherein the filter member further comprises a body having a first end, a second end, and a sidewall extending from the first end to the second end, the sidewall of the body defining a passageway extending through the body from the first end to the second end, and wherein the gas-permeable, liquid-impermeable membrane extends across an entirety of the passageway.

The illustrative combination wherein the gas-permeable, liquid-impermeable membrane is secured to the body adjacent the second end of the body.

The illustrative combination wherein the filter member is slidably disposed in the internal chamber of the barrel.

The illustrative combination wherein the sidewall of the body has at least two annular projections extending radially outwardly in slidable, sealing contact with the inner surface of the barrel.

The illustrative combination wherein the gas-permeable, liquid-impermeable membrane is formed from a material comprising at least one of polytetraflyorethylene, polypropylene, and polyethylene.

The illustrative combination wherein the body has a plurality of passageways extending through the body from the first end to the second end, and wherein the filter member further has a plurality of gas-permeable, liquid impermeable membranes with at least one of the gas-permeable, liquid impermeable membranes extending across each of the passageways of the body.

The illustrative combination wherein the filter member further comprises a plurality of porous filter material positioned between the first end of the body and each of the gas-permeable, liquid impermeable membranes to prevent solid particulate from contacting the gas-permeable, liquid impermeable membranes.

The illustrative combination wherein the plurality of gas-permeable, liquid-impermeable membranes are secured to the body adjacent the second end of the body.

The illustrative combination wherein the passageways are in a parallel relationship to one another.

An illustrative method for removing bubbles from a fluid sample having a liquid portion and a gas portion, comprising:
receiving a flow of the fluid sample from an inlet opening of a barrel having a first end, a second end, a sidewall extending between the first end and the second end, and an inner surface defining an internal chamber, the first end having the inlet opening and the second end having an outlet opening a first substrate having a first side, a second side opposite the first side, and an opening extending through the first substrate from the first side to the second side;
passing the fluid sample into the internal chamber via the inlet opening;
causing the fluid sample to contact a filter member disposed within the internal chamber, the filter member defining an inlet side and an outlet side of the internal chamber, the filter member having a gas-permeable, liquid-impermeable membrane;
separating at least a portion of the gas portion of the liquid sample from the liquid portion of the fluid sample by causing the fluid sample to contact the gas-permeable, liquid-impermeable membrane so that at least a portion of the gas portion of the fluid sample passes across the filter member from the inlet side to the outlet side of the internal chamber and so the liquid portion of the fluid sample is prevented from passing from the inlet side to the outlet side;
collecting at least a portion of the liquid portion of the fluid sample from the inlet side of the internal chamber.

The illustrative method wherein at least a portion of the liquid portion of the fluid sample is collected from the inlet side of the internal chamber by piercing the filter member so a probe may be passed through the filter member from the outlet side to the inlet side to withdraw the liquid portion of the fluid sample from the inlet side of the internal chamber.

The illustrative method wherein the filter member further comprises a filter element interposed between the gas-permeable, liquid-impermeable membrane and the inlet side of the internal chamber, the at least one filter element having an inlet port and an outlet port, the inlet port in fluid communication with the inlet side of the internal chamber, the outlet port in fluid communication with the gas-permeable, liquid-impermeable membrane to permit at least a portion of the fluid sample to pass across the at least one filter element and to the gas-permeable, liquid-impermeable membrane.

The illustrative method wherein the gas-permeable, liquid-impermeable membrane is a plurality of gas-permeable, liquid-impermeable membranes.

What is claimed:

1. An apparatus for removing bubbles from a fluid sample having a liquid portion and a gas portion, comprising:
a barrel having a first end, a second end, a sidewall extending between the first end and the second end, and an inner surface defining an internal chamber, the first end having an inlet opening and the second end having an outlet opening; and first substrate having a first side, a second side opposite the first side, and an opening extending through the first substrate from the first side to the second side; and
a filter member disposed within the internal chamber so the filter member defines an inlet side and an outlet side of the internal chamber and so the filter member is positionable between the first end and the second end of the barrel, the filter member having at least one gas-permeable, liquid-impermeable membrane to permit at least a portion of the gas portion of the fluid sample to pass across the filter member from the inlet side to the outlet side of the internal chamber and to provide a fluid-tight seal across the filter member to prevent the liquid portion of the fluid sample from passing from the inlet side to the outlet side as the fluid sample is passed into the internal chamber via the inlet opening to separate at least a portion of the gas portion from the liquid portion of the fluid sample;
wherein the filter member is pierceable so a probe may be passed through the filter member from the outlet side to the inlet side to withdraw the liquid portion of the fluid sample from the inlet side of the internal chamber.

2. The apparatus of claim 1, wherein the filter member further comprises a body having a first end, a second end, and a sidewall extending from the first end to the second end, the sidewall of the body defining a passageway extending through the body from the first end to the second end, and wherein the gas-permeable, liquid-impermeable membrane extends across an entirety of the passageway.

3. The apparatus of claim 2, wherein the gas-permeable, liquid-impermeable membrane is secured to the body adjacent the second end of the body.

4. The apparatus of claim 1, wherein the filter member is slidably disposed in the internal chamber of the barrel.

5. The apparatus of claim 4, wherein the sidewall of the body has at least two annular projections extending radially outwardly in slidable, sealing contact with the inner surface of the barrel.

6. The apparatus of claim 1, wherein the gas-permeable, liquid-impermeable membrane is formed from a material comprising at least one of polytetraflyorethylene, polypropylene, and polyethylene.

7. The apparatus of claim 2, wherein the body has a plurality of passageways extending through the body from the first end to the second end, and wherein the filter member further has a plurality of gas-permeable, liquid impermeable membranes with at least one of the gas-permeable, liquid impermeable membranes extending across each of the passageways of the body.

8. The apparatus of claim 7, wherein the filter member further comprises a plurality of porous filter material positioned between the first end of the body and each of the gas-permeable, liquid impermeable membranes to prevent solid particulate from contacting the gas-permeable, liquid impermeable membranes.

9. The apparatus of claim 7, wherein the plurality of gas-permeable, liquid-impermeable membranes are secured to the body adjacent the second end of the body.

10. The apparatus of claim 7, wherein the passageways are in a parallel relationship to one another.

11. An apparatus for removing bubbles from a fluid sample having a liquid portion and a gas portion in combination with a liquid sample analyzer having a sample probe, the apparatus comprising:
a barrel having a first end, a second end, a sidewall extending between the first end and the second end, and an inner surface defining an internal chamber, the first end having an inlet opening and the second end being connected to the liquid sample analyzer and having an outlet opening; and
a filter member disposed within the internal chamber so the filter member defines an inlet side and an outlet side of the internal chamber and so the filter member is positionable between the first end and the second end of the barrel, the filter member having at least one gas-permeable, liquid-impermeable membrane to permit at least a portion of the gas portion of the fluid sample to pass across the filter member from the inlet side to the outlet side of the internal chamber and to provide a fluid-tight seal across the filter member to prevent the liquid portion of the fluid sample from passing from the inlet side to the outlet side as the fluid sample is passed into the internal chamber via the inlet opening to separate at least a portion of the gas portion from the liquid portion of the fluid sample;
wherein the filter member is pierceable by the sample probe so the sample probe may be passed through the outlet opening of the barrel and through the filter member from the outlet side to the inlet side to draw the liquid portion of the fluid sample into the liquid sample analyzer from the inlet side of the internal chamber.

12. The combination of claim 11, wherein the filter member further comprises a body having a first end, a second end, and a sidewall extending from the first end to the second end, the sidewall of the body defining a passageway extending through the body from the first end to the second end, and wherein the gas-permeable, liquid-impermeable membrane extends across an entirety of the passageway.

13. The combination of claim 12, wherein the gas-permeable, liquid-impermeable membrane is secured to the body adjacent the second end of the body.

14. The combination of claim 11, wherein the filter member is slidably disposed in the internal chamber of the barrel.

15. The combination of claim 14, wherein the sidewall of the body has at least two annular projections extending radially outwardly in slidable, sealing contact with the inner surface of the barrel.

16. The combination of claim 14, wherein the gas-permeable, liquid-impermeable membrane is formed from a material comprising at least one of polytetraflyorethylene, polypropylene, and polyethylene.

17. The combination of claim 12, wherein the body has a plurality of passageways extending through the body from the first end to the second end, and wherein the filter member further has a plurality of gas-permeable, liquid impermeable membranes with at least one of the gas-permeable, liquid impermeable membranes extending across each of the passageways of the body.

18. The combination of claim 17, wherein the filter member further comprises a plurality of porous filter material positioned between the first end of the body and each of the gas-permeable, liquid impermeable membranes to prevent solid particulate from contacting the gas-permeable, liquid impermeable membranes.

19. The combination of claim 17, wherein the plurality of gas-permeable, liquid-impermeable membranes are secured to the body adjacent the second end of the body.

20. The combination of claim 17, wherein the passageways are in a parallel relationship to one another.

21. A method of removing bubbles from a fluid sample having a liquid portion and a gas portion, the method comprising:
receiving a flow of the fluid sample from an inlet opening of a barrel having a first end, a second end, a sidewall extending between the first end and the second end, and an inner surface defining an internal chamber, the first end having the inlet opening and the second end having an outlet opening;
passing the fluid sample into the internal chamber via the inlet opening;
causing the fluid sample to contact a filter member disposed within the internal chamber, the filter member defining an inlet side and an outlet side of the internal chamber, the filter member having a gas-permeable, liquid-impermeable membrane;
separating at least a portion of the gas portion of the liquid sample from the liquid portion of the fluid sample by causing the fluid sample to contact the gas-permeable, liquid-impermeable membrane so that at least a portion of the gas portion of the fluid sample passes across the filter member from the inlet side to the outlet side of the internal chamber and so the liquid portion of the fluid sample is prevented from passing from the inlet side to the outlet side;
collecting at least a portion of the liquid portion of the fluid sample from the inlet side of the internal chamber.

22. The method of claim 21, wherein at least a portion of the liquid portion of the fluid sample is collected from the inlet side of the internal chamber by piercing the filter member so a probe may be passed through the filter member from the outlet side to the inlet side to withdraw the liquid portion of the fluid sample from the inlet side of the internal chamber.

23. The method of claim 21, wherein the filter member further comprises a filter element interposed between the gas-permeable, liquid-impermeable membrane and the inlet side of the internal chamber, the at least one filter element having an inlet port and an outlet port, the inlet port in fluid communication with the inlet side of the internal chamber, the outlet port in fluid communication with the gas-permeable, liquid-impermeable membrane to permit at least a portion of the fluid sample to pass across the at least one filter element and to the gas-permeable, liquid-impermeable membrane.

24. The method of claim 21, wherein the gas-permeable, liquid-impermeable membrane is a plurality of gas-permeable, liquid-impermeable membranes.
